(19) United States
(12) Patent Application Publication
(10) Pub. No.: US 2008/0268000 A1
(43) Pub. Date: Oct. 30, 2008

(54) Vaccine Stabilising Formula with Live Antigens for Use in Mass Vaccination Systems

(76) Inventor: Jose Luis Nuno Ayala, Jalisco (MX)

Correspondence Address: BANNER & WITCOFF, LTD., 1100 13th STREET, N.W., SUITE 1200, WASHINGTON, DC, 20005-4051, US

(21) Appl. No.: 12/065,216

(22) PCT Filed: Sep. 1, 2005
(86) PCT No.: PCT/MX2005/000073

§ 371 Date: Jul. 14, 2008

(51) Int. Cl. A61K 47/00

(52) U.S. Cl. 424/278.1

(57) ABSTRACT

The invention relates to a vaccine stabilizing formula with live antigens for use in mass medication and vaccination systems which are used in animal protein production processes. The invention comprises a product in the form of a powder or liquid to enable the same to be solubilized in water formed by a homogeneous mixture of compounds of organic and inorganic origin. The invention can be used to stabilize the critical physicochemical parameters of the water upon vaccination (pH, hardness and presence of disinfectants), thereby reducing the microbial and viral liter loss of the vaccine caused by environmental exposure upon vaccination. The inventive formula is intended to be used in drinking water medication and vaccination systems and in spray medication and vaccination systems.

### BACKGROUND OF THE INVENTION

The invention herein disclosed is a live vaccine stabilizing formula for increasing the solution viability time thereof and thus achieve the correct immunization of animals. The preparation and application method is also disclosed.

Vaccination, as a prophylactic measure, is part of a series of activities directed towards achieving health and well-being of animals in modern production systems. Together with biosecurity systems and an adequate diet, it allows the producer to achieve a correct performance of the productive system during the whole cycle and therefore a higher economical profit. The importance of an adequate vaccination program becomes evident by observing the significant economical losses caused by an infectious outbreak brought about by a low ability of animals to control a field challenge.

Several factors have to be analyzed when defining a vaccination program, among which there are the kind of vaccine to be used, the strain, the administration route, the technique to use and the follow up methodologies.

Within the technical aspects, the application route and the technique with which to apply are highly important, as their execution directly defines the possible success or failure of the operation.

The vaccine by itself does not represent one of the higher costs in an operation, however, it is one of the activities that requires more attention, logistics and supervision. The cost of manpower related to this activity is high as are the error possibilities, deviations or low uniformity.

In the case of live antigen vaccines, the handling and care that have to be taken for a successful vaccination are specially critical. The current intensive production systems need vaccination and medication massive techniques that allow the producer to immunize a great deal of poultry in a short time, using low manpower, in order to lower the associated costs and raising the gain margin.

These systems must also provide for flexibility and security to the producer in order to face the new health challenges caused by an increase in contagious diseases as a result of an increase in poultry density of the new productive systems.

Considering the magnitude of poultry-farming exploitations, the water quality used in production farms is hard to assure, due to the presence of pathogen microorganisms and adverse physiochemical conditions. Depending on the geographical location of the water supply used and its type of storage, it can have variable hardness (mineral calcium and magnesium) conditions, far from neutral pH ranges, and having halogen, chloride and iodide presence, coming from treatment systems for eliminating water pathogens.

These conditions are adverse for animal health, but they are even worse for vaccination and medication procedures thereof. Vaccination and medication are two procedures that require very specific water conditions: a neutral pH, absence of chlorine and iodine, low hardness, and therefore, rarely optimal results can be achieved at normal field conditions.

However, in order for this operation to provide maximum possible protection, some factors have to be taken care of, among them and most important being the quality of water to be sprayed. Using water which does not have the minimum quality features for vaccination irreversibly reduces the efficiency of the vaccine used, thus risking the health of the vaccinated flock.

The minimum quality conditions required for achieving maximum efficiency of vaccination and medication processes are related to three main parameters: pH, water hardness and the presence of halogens as disinfectants.

pH Is a determining factor for the efficiency of vaccination and medication procedures. In case of the former, viruses and bacteria are highly resistant to denaturalization or inactivation by higher than neutral acidity or alkalinity ranges. The ideal pH for vaccination or medication is pH 7.

Water hardness means the presence of calcium and magnesium ions, mainly coming from underground ore deposits. These ions have chelating or agglutinating functions of the viruses, bacteria and active principles used in prophylaxis. The ideal condition is an absence or minimal concentration possible of these ions in water when vaccinating or medicating.

Due to the presence of pathogens in water, it is needed to treat it with antimicrobials, among the most widely used are chlorine and iodine. However, they interfere with the vaccination processes as they have the same effect on the vaccine thus inactivating it. For this operation, water needs to be completely halogen-free.

Media conditions present in poultry digestive and respiratory tracts adversely affect viruses and bacteria viability when vaccinating. Thus the use of some resource that prevents the destruction of antigens that will be supplied before they settle on the specific tissue over which they must grow.

Every live microorganism, whether viruses or bacteria, has a spatial structure, or tertiary structure, which is modified by the osmotic pressure of the solution, and which when altered could cause disruption of bacteria outer walls or the denaturalization of most of the viruses. Hence, it is ideal for the vaccination solution to have minimal isoelectric potential in order to keep the live antigens stable.

A common and widely spread practice several years ago in the livestock industry is the use of skimmed milk as a stabilizer for live antigen vaccines. Due to the slightly basic features of milk, it is attributed pH neutralizing qualities.

The use of milk as a stabilizer is not very convenient since in many occasions milk itself contains iodide residues that breakdown the vaccine, therefore only the use of milk that could be certified as iodide-free or commercial stabilizing preparations must be considered. Still, milk's ability to stabilize pH and halogens presence in water is very limited, almost null.

It also poses very serious problems because of its calcium content, as it substantially raises water hardness levels and therefore could affect both the stability of the antigen and can also cause calcium deposits on water feeding systems piping or on sprayer nozzles. It is also a culture medium causing bioplaque film formation inside water distribution systems or spray systems that turn into very strong risks for animal health.

It is therefore necessary to have a product which provides the necessary stabilization of antigen in solution by addressing all the three aforesaid conditions, which possess a high stabilizing efficiency, immediate action and at the same time not affecting the mechanical systems used for vaccination.

### DISCLOSURE OF THE INVENTION

The application field of the invention is in drinking water vaccination and medication systems and spray vaccination and medication for live antigen vaccines. The vaccine distribution medium is commonly water, but is not limited thereto, so the vaccine can be applied in other distribution medium, as long as it is a liquid medium.

The stabilizing formula for vaccines with live antigens for use in massive vaccination systems comprises a mixture of ingredients formulated to confer extended stability to live antigens (live virus or bacteria vaccines) used in animal vaccination for animal production processes.

The invention comprises a product which can be in liquid or solid form for being mixed in the water used for farm animal vaccination.

A pH stabilizing agent will be used in order to stabilize the pH of the vaccine solution at a range of 6.5 to 7.5, range in which most of bacteria and viruses are viable. The pH stabilizing agents can be phosphates, succinates, bicarbonates and lactates. It is preferred the use of potassium phosphates due to their low irritating potential and as they are considered as safe for use with animals and human beings.

A water hardness sequestrant agent will be used for removing minerals dissolved in water which could cause the inactivation of the viruses and bacteria present in the vaccine. Among the sequestrant agents there are salts of ethylene diamine tetraacetic acid. Among them it is preferred to use ethylene diaminetetraacetic acid disodium salt (disodium EDTA) because it is considered safe to use, it does not significantly modifies the solution pH and causes no irritation in live tissues.

A reducing agent is used for neutralizing the sanitizers present in local water where the application is to take place. These sanitizers can be chlorine-, iodine-, peroxide-, bromine-, fluorine-, ozone- or permanganate-based. All these can be considered as oxidant agents that can be neutralized through, but not limited to, sodium thiosulfate, sodium metabisulfite, sodium bisulfite, sodium sulfite, sulphur dioxide, ammonium bisulfite, and ammonium thiosulfate. Use of sodium thiosulfate is preferred as it possess a high neutralization ability and is considered safe and not corrosive.

When necessary, a water soluble salt will be used in order to provide an osmotic pressure needed for keeping stable the tertiary structure of viruses and intact the outer wall of bacteria present in the vaccine. Among them there are chlorides, iodides, carbonates, bicarbonates, phosphates, iodates, chlorates, bromides, bromates, fluorides, nitrates, nitrites, sulfides, sulfates and sulfites. From these use is preferred of sodium chloride due to its null toxicity, irritability and a it is considered safe for its consumption.

When necessary, a carbohydrate shall be used for protecting virus or bacterium structure from the attack of poultry digestive tract adverse conditions, and it can be among others, glucose, dextrose, lactose, sucrose, mannose and fructose. Lactose is preferred as it possess high solubility due to its dust particle size and as it displays no negative interaction with the normal metabolism of poultry.

When necessary, a food grade colorant will be used for promoting a visual verification medium for the vaccinator, which will allow the user of the stabilizer to know when the protector has been added to water, and eventually, that the animal has already received the vaccine carrier water. Among them there are colors and blue, red, green, violet, orange, etc. color preparations. From them, the use of blue color is preferred due to its contrasting effects on the color of live tissues.

When necessary, a food grade anti-wetting agent will be used in order to prevent wetting of the mixture as it has hygroscopic properties in some of its salts. These agents do not modify the product's physiochemical functioning and only work to reduce wetting of the mixture. Examples of them are silicon dioxide, calcium stearate, magnesium stereate, tribasic calcium phosphate, tribasic magnesium phosphate, magnesium oxide, calcium silicate, magnesium silicate, sodium silico aluminate, calcium silicate aluminate, etc. From these use is preferred of magnesium stearate and silicone dioxide due to their null toxicity, irritability and because they are considered safe for consumption.

The formulation is made up in the following way: ethylene diaminetetraacetic acid (EDTA) disodium salt at a range of 0.03% to 34.19% with an optimal concentration of 3.75%, monobasic potassium phosphate at a range of 0.03% to 34.48% with an optimal concentration of 5%, dibasic potassium phosphate at a range of 0.03% to 56.07% with an optimal concentration of 41.25%, sodium thiosulfate at a range of 0.03% to 33.73% with an optimal concentration of 1.75%, sodium chloride at a range of 0.03% to 35.91% with an optimal concentration of 10.75%, lactose at a range of 0.03% to 51.02% with an optimal concentration of 28%, silicon dioxide at a range of 0.03% to 5% with an optimal concentration of 1%, magnesium stearate at a range of 0.03% to 5% with an optimal concentration of 1% and brilliant blue colorant at a range of 0.03% to 44.25% with an optimal concentration of 7.50%.

The manufacturing process is formed by the progressive mixing of these raw materials under constant agitation for 15 minutes between each addition, at a maximum relative humidity of 30% and a temperature in the range of 15° C. to 35° C., in the following order: dibasic potassium phosphate, lactose, monobasic potassium phosphate, ethylene diaminetetraacetic acid (EDTA) disodium salt, sodium thiosulfate, sodium chloride, magnesium stearate, silicon dioxide and brilliant blue color.

When mixing the vaccine with the stabilizing solution at a concentration between 2.85 g/L and 5.93 g/l of solution to be prepared at a temperature between 15° C. and 35° C., the virus or medicament is not exposed to adverse conditions so that its viability is preserved for long periods of time. When animals drink the solution with the vaccine, lactose works as a protector on the virus outer surface, thus avoiding it to be damaged during its transit up to reaching the lower digestive system, wherein the vaccine passes to the bloodstream through the intestine membranes and starts the immunogenic reaction.

The use procedure is defined in the following way: for each vaccine solution liter to be prepared there will be added, depending on the composition, an amount in the range of 2.85 to 5.93 g/L of the live vaccine stabilizer slowly adding with continuous agitation. Once solubilized the required amount is added to the vaccine and applied to the animals to be vaccinated.

### EXAMPLE 1

A powder mixture containing 0.03% EDTA, 5.19% monobasic potassium phosphate, 42.85% dibasic potassium phosphate, 1.82% sodium thiosulfate, 11.17% sodium chloride, 31.16% lactose, 1.04% silicon dioxide, 1.04% magnesium stearate and 7.79% colorant. This mixture is metered at a ratio of 3.85 g per each liter of water to prepare.

A laboratory scale test was conducted in which the product was applied to a water blank solution having pH 6, 100 ppm total hardness and 5 ppm free chlorine, and within five minutes a vial having 1000 doses with a titer of 3.7 log 10 of vaccine against avian infectious bronchitis disease which was used as biological model (sample A) and a duplicate sample with the blank solution without the product (sample B) was also applied.

After 2 hours of mixing the following physiochemical measurements by instrumental analysis and biological measurements by chick embryo titration were obtained:

Sample A: water hardness of 55 ppm total hardness, a pH of 7.1 and a zero free chlorine titration. It was observed that the vaccine titer was kept at a concentration of 94% of the initial applied concentration of the vaccine so that there was only 6% of vaccine activity loss.

Sample B: a hardness of 115 ppm, pH of 7.1 and 4 ppm of free chlorine were obtained. It was observed that the vaccine titer was reduced to 30% of the initial applied concentration of the vaccine so there was a 70% vaccine activity loss.

### EXAMPLE 2

A powder mixture containing 3.75% EDTA, 5.00% monobasic potassium phosphate, 41.25% dibasic potassium phosphate, 1.75% sodium thiosulfate, 10.75% sodium chloride, 28% lactose, 1% magnesium stearate, 1% silicon dioxide and 7.50% colorant. This mixture is metered at a ratio of 4 g per each liter of water to prepare.

A laboratory scale test was conducted in which the product was applied to a water blank solution having pH 6, 100 ppm total hardness and 5 ppm free chlorine, and within five minutes a vial having 1000 doses with a titer of 3.7 log 10 of vaccine against avian infectious bronchitis disease which was used as biological model (sample A) and a duplicate sample with the blank solution without the product (sample B) was also applied.

After 2 hours of mixing the following physiochemical measurements by instrumental analysis and biological measurements by chick embryo titration were obtained:

Sample A: water hardness of 0 ppm total hardness, a pH of 7.0 and a zero free chlorine titration. It was observed that the vaccine titer was kept at a concentration of 97% of the initial applied concentration of the vaccine so that there was only 3% of vaccine activity loss.

Sample B: a hardness of 105 ppm, pH of 5.9 and 5 ppm of free chlorine were obtained. It was observed that the vaccine titer was reduced to 25% of the initial applied concentration of the vaccine so there was a 75% vaccine activity loss.

### EXAMPLE 3

A powder mixture containing 34.19% EDTA, 3.42% monobasic potassium phosphate, 28.21% dibasic potassium phosphate, 1.20% sodium thiosulfate, 7.35% sodium chloride, 19.15% lactose, 0.68% magnesium stearate, 0.68% silicon dioxide and 5.13% colorant. This mixture is metered at a ratio of 5.85 g per each liter of water to prepare.

A laboratory scale test was conducted in which the product was applied to a water blank solution having pH 6, 100 ppm total hardness and 5 ppm free chlorine, and within five minutes a vial having 1000 doses with a titer of 3.7 log 10 of vaccine against avian infectious bronchitis disease which was used as biological model (sample A) and a duplicate sample with the blank solution without the product (sample B) was also applied.

After 2 hours of mixing the following physiochemical measurements by instrumental analysis and biological measurements by chick embryo titration were obtained:

Sample A: water hardness of 0 ppm total hardness, a pH of 7.0 and a zero free chlorine titration. It was observed that the vaccine titer was kept at a concentration of 97% of the initial applied concentration of the vaccine so that there was only 3% of vaccine activity loss.

Sample B: a hardness of 110 ppm, pH of 6.0 and 4 ppm of free chlorine were obtained. It was observed that the vaccine titer was reduced to 30% of the initial applied concentration of the vaccine so there was a 70% vaccine activity loss.

### EXAMPLE 4

A powder mixture containing 3.95% EDTA, 0.03% monobasic potassium phosphate, 43.41% dibasic potassium phosphate, 1.84% sodium thiosulfate, 11.31% sodium chloride, 29.47% lactose, 1.05% magnesium stearate, 1.05% silicon dioxide and 7.89% colorant. This mixture is metered at a ratio of 3.8 g per each liter of water to prepare.

A laboratory scale test was conducted in which the product was applied to a water blank solution having pH 6, 100 ppm total hardness and 5 ppm free chlorine, and within five minutes a vial having 1000 doses with a titer of 3.7 log 10 of vaccine against avian infectious bronchitis disease which was used as biological model (sample A) and a duplicate sample with the blank solution without the product (sample B) was also applied.

After 2 hours of mixing the following physiochemical measurements by instrumental analysis and biological measurements by chick embryo titration were obtained:

Sample A: water hardness of 0 ppm total hardness, a pH of 7.6 and a zero free chlorine titration. It was observed that the vaccine titer was kept at a concentration of 93% of the initial applied concentration of the vaccine so that there was only 7% of vaccine activity loss.

Sample B: a hardness of 102 ppm, pH of 6.0 and 5 ppm of free chlorine were obtained. It was observed that the vaccine titer was reduced to 35% of the initial applied concentration of the vaccine so there was a 65% vaccine activity loss.

### EXAMPLE 6

A powder mixture containing 2.59% EDTA, 34.48% monobasic potassium phosphate, 28.45% dibasic potassium phosphate, 1.21% sodium thiosulfate, 7.41% sodium chloride, 19.31% lactose, 0.69% magnesium stearate, 0.69% silicon dioxide and 5.17% colorant. This mixture is metered at a ratio of 5.8 g per each liter of water to prepare.

A laboratory scale test was conducted in which the product was applied to a water blank solution having pH 6, 100 ppm total hardness and 5 ppm free chlorine, and within five minutes a vial having 1000 doses with a titer of 3.7 log 10 of vaccine against avian infectious bronchitis disease which was used as biological model (sample A) and a duplicate sample with the blank solution without the product (sample B) was also applied.

After 2 hours of mixing the following physiochemical measurements by instrumental analysis and biological measurements by chick embryo titration were obtained:

Sample A: water hardness of 0 ppm total hardness, a pH of 6.2 and a zero free chlorine titration. It was observed that the vaccine titer was kept at a concentration of 83% of the initial applied concentration of the vaccine so that there was only 17% of vaccine activity loss.

Sample B: a hardness of 104 ppm, pH of 6.0 and 5 ppm of free chlorine were obtained. It was observed that the vaccine titer was reduced to 33% of the initial applied concentration of the vaccine so there was a 67% vaccine activity loss.

### EXAMPLE 7

A powder mixture containing 6.32% EDTA, 8.51% monobasic potassium phosphate, 0.03% dibasic potassium phosphate, 2.98% sodium thiosulfate, 18.29% sodium chloride, 47.64% lactose, 1.70% magnesium stearate, 1.70% silicon dioxide and 12.76% colorant. This mixture is metered at a ratio of 2.35 g per each liter of water to prepare.

A laboratory scale test was conducted in which the product was applied to a water blank solution having pH 6, 100 ppm total hardness and 5 ppm free chlorine, and within five minutes a vial having 1000 doses with a titer of 3.7 log 10 of vaccine against avian infectious bronchitis disease which was used as biological model (sample A) and a duplicate sample with the blank solution without the product (sample B) was also applied.

After 2 hours of mixing the following physiochemical measurements by instrumental analysis and biological measurements by chick embryo titration were obtained:

Sample A: water hardness of 2 ppm total hardness, a pH of 5.8 and a zero free chlorine titration. It was observed that the vaccine titer was kept at a concentration of 73% of the initial applied concentration of the vaccine so that there was only 27% of vaccine activity loss.

Sample B: a hardness of 102 ppm, pH of 5.9 and 4 ppm of free chlorine were obtained. It was observed that the vaccine titer was reduced to 29% of the initial applied concentration of the vaccine so there was a 71% vaccine activity loss.

### EXAMPLE 8

A powder mixture containing 2.80% EDTA, 3.74% monobasic potassium phosphate, 56.07% dibasic potassium phosphate, 1.31% sodium thiosulfate, 8.04% sodium chloride, 20.93% lactose, 0.75% magnesium stearate, 0.75% silicon dioxide and 5.61% colorant. This mixture is metered at a ratio of 5.35 g per each liter of water to prepare.

A laboratory scale test was conducted in which the product was applied to a water blank solution having pH 6, 100 ppm total hardness and 5 ppm free chlorine, and within five minutes a vial having 1000 doses with a titer of 3.7 log 10 of vaccine against avian infectious bronchitis disease which was used as biological model (sample A) and a duplicate sample with the blank solution without the product (sample B) was also applied.

After 2 hours of mixing the following physiochemical measurements by instrumental analysis and biological measurements by chick embryo titration were obtained:

Sample A: water hardness of 3 ppm total hardness, a pH of 7.6 and a zero free chlorine titration. It was observed that the vaccine titer was kept at a concentration of 91% of the initial applied concentration of the vaccine so that there was only 9% of vaccine activity loss.

Sample B: a hardness of 108 ppm, pH of 6.1 and 5 ppm of free chlorine were obtained. It was observed that the vaccine titer was reduced to 36% of the initial applied concentration of the vaccine so there was a 64% vaccine activity loss.

### EXAMPLE 9

A powder mixture containing 3.82% EDTA, 5.09% monobasic potassium phosphate, 41.97% dibasic potassium phosphate, 0.03% sodium thiosulfate, 10.94% sodium chloride, 28.49% lactose, 1.02% magnesium stearate, 1.02% silicon dioxide and 7.63% colorant. This mixture is metered at a ratio of 3.93 g per each liter of water to prepare.

A laboratory scale test was conducted in which the product was applied to a water blank solution having pH 6, 100 ppm total hardness and 5 ppm free chlorine, and within five minutes a vial having 1000 doses with a titer of 3.7 log 10 of vaccine against avian infectious bronchitis disease which was used as biological model (sample A) and a duplicate sample with the blank solution without the product (sample B) was also applied.

After 2 hours of mixing the following physiochemical measurements by instrumental analysis and biological measurements by chick embryo titration were obtained:

Sample A: water hardness of 4 ppm total hardness, a pH of 7.1 and a 2 ppm free chlorine titration. It was observed that the vaccine titer was kept at a concentration of 72% of the initial applied concentration of the vaccine so that there was only 28% of vaccine activity loss.

Sample B: a hardness of 100 ppm, pH of 6.0 and 5 ppm of free chlorine were obtained. It was observed that the vaccine titer was reduced to 35% of the initial applied concentration of the vaccine so there was a 65% vaccine activity loss.

### EXAMPLE 10

A powder mixture containing 2.53% EDTA, 3.37% monobasic potassium phosphate, 27.82% dibasic potassium phosphate, 33.73% sodium thiosulfate, 7.25% sodium chloride, 18.89% lactose, 0.67% magnesium stearate, 0.67% silicon dioxide and 5.06% colorant. This mixture is metered at a ratio of 5.93 g per each liter of water to prepare.

A laboratory scale test was conducted in which the product was applied to a water blank solution having pH 6, 100 ppm total hardness and 5 ppm free chlorine, and within five minutes a vial having 1000 doses with a titer of 3.7 log 10 of vaccine against avian infectious bronchitis disease which was used as biological model (sample A) and a duplicate sample with the blank solution without the product (sample B) was also applied.

After 2 hours of mixing the following physiochemical measurements by instrumental analysis and biological measurements by chick embryo titration were obtained:

Sample A: water hardness of 1 ppm total hardness, a pH of 7.0 and a zero free chlorine titration. It was observed that the vaccine titer was kept at a concentration of 96% of the initial applied concentration of the vaccine so that there was only 4% of vaccine activity loss.

Sample B: a hardness of 107 ppm, pH of 6.1 and 5 ppm of free chlorine were obtained. It was observed that the vaccine titer was reduced to 32% of the initial applied concentration of the vaccine so there was a 68% vaccine activity loss.

### EXAMPLE 11

A powder mixture containing 4.20% EDTA, 5.60% monobasic potassium phosphate, 46.21% dibasic potassium phosphate, 1.96% sodium thiosulfate, 0.03% sodium chloride, 31.36% lactose, 1.12% magnesium stearate, 1.12% silicon dioxide and 8.40% colorant. This mixture is metered at a ratio of 3.57 g per each liter of water to prepare.

A laboratory scale test was conducted in which the product was applied to a water blank solution having pH 6, 100 ppm total hardness and 5 ppm free chlorine, and within five minutes a vial having 1000 doses with a titer of 3.7 log 10 of vaccine against avian infectious bronchitis disease which was used as biological model (sample A) and a duplicate sample with the blank solution without the product (sample B) was also applied.

After 2 hours of mixing the following physiochemical measurements by instrumental analysis and biological measurements by chick embryo titration were obtained:

Sample A: water hardness of 0 ppm total hardness, a pH of 7.2 and a zero free chlorine titration. It was observed that the vaccine titer was kept at a concentration of 96% of the initial applied concentration of the vaccine so that there was only 4% of vaccine activity loss.

Sample B: a hardness of 103 ppm, pH of 6.0 and 5 ppm of free chlorine were obtained. It was observed that the vaccine titer was reduced to 28% of the initial applied concentration of the vaccine so there was a 72% vaccine activity loss.

### EXAMPLE 12

A powder mixture containing 2.69% EDTA, 3.59% monobasic potassium phosphate, 29.62% dibasic potassium phosphate, 1.26% sodium thiosulfate, 35.91% sodium chloride, 20.11% lactose, 0.72% magnesium stearate, 0.72% silicon dioxide and 5.39% colorant. This mixture is metered at a ratio of 5.57 g per each liter of water to prepare.

A laboratory scale test was conducted in which the product was applied to a water blank solution having pH 6, 100 ppm total hardness and 5 ppm free chlorine, and within five minutes a vial having 1000 doses with a titer of 3.7 log 10 of vaccine against avian infectious bronchitis disease which was used as biological model (sample A) and a duplicate sample with the blank solution without the product (sample B) was also applied.

After 2 hours of mixing the following physiochemical measurements by instrumental analysis and biological measurements by chick embryo titration were obtained:

Sample A: water hardness of 3 ppm total hardness, a pH of 6.8 and a zero free chlorine titration. It was observed that the vaccine titer was kept at a concentration of 91% of the initial applied concentration of the vaccine so that there was only 9% of vaccine activity loss.

Sample B: a hardness of 100 ppm, pH of 6.0 and 5 ppm of free chlorine were obtained. It was observed that the vaccine titer was reduced to 31% of the initial applied concentration of the vaccine so there was a 69% vaccine activity loss.

### EXAMPLE 13

A powder mixture containing 5.21% EDTA, 6.94% monobasic potassium phosphate, 57.27% dibasic potassium phosphate, 2.43% sodium thiosulfate, 14.93% sodium chloride, 0.03% lactose, 1.39% magnesium stearate, 1.39% silicon dioxide and 10.41% colorant. This mixture is metered at a ratio of 2.88 g per each liter of water to prepare.

A laboratory scale test was conducted in which the product was applied to a water blank solution having pH 6, 100 ppm total hardness and 5 ppm free chlorine, and within five minutes a vial having 1000 doses with a titer of 3.7 log 10 of vaccine against avian infectious bronchitis disease which was used as biological model (sample A) and a duplicate sample with the blank solution without the product (sample B) was also applied.

After 2 hours of mixing the following physiochemical measurements by instrumental analysis and biological measurements by chick embryo titration were obtained:

Sample A: water hardness of 4 ppm total hardness, a pH of 7.0 and a zero free chlorine titration. It was observed that the vaccine titer was kept at a concentration of 93% of the initial applied concentration of the vaccine so that there was only 7% of vaccine activity loss.

Sample B: a hardness of 100 ppm, pH of 5.9 and 4 ppm of free chlorine were obtained. It was observed that the vaccine titer was reduced to 36% of the initial applied concentration of the vaccine so there was a 64% vaccine activity loss.

### EXAMPLE 14

A powder mixture containing 2.55% EDTA, 3.40% monobasic potassium phosphate, 28.06% dibasic potassium phosphate, 1.19% sodium thiosulfate, 7.31% sodium chloride, 51.02% lactose, 0.68% magnesium stearate, 0.68% silicon dioxide and 5.1% colorant. This mixture is metered at a ratio of 6.88 g per each liter of water to prepare.

A laboratory scale test was conducted in which the product was applied to a water blank solution having pH 6, 100 ppm total hardness and 5 ppm free chlorine, and within five minutes a vial having 1000 doses with a titer of 3.7 log 10 of vaccine against avian infectious bronchitis disease which was used as biological model (sample A) and a duplicate sample with the blank solution without the product (sample B) was also applied.

After 2 hours of mixing the following physiochemical measurements by instrumental analysis and biological measurements by chick embryo titration were obtained:

Sample A: water hardness of 21 ppm total hardness, a pH of 7.3 and a zero free chlorine titration. It was observed that the vaccine titer was kept at a concentration of 95% of the initial applied concentration of the vaccine so that there was only 5% of vaccine activity loss.

Sample B: a hardness of 107 ppm, pH of 6.0 and 5 ppm of free chlorine were obtained. It was observed that the vaccine titer was reduced to 31% of the initial applied concentration of the vaccine so there was a 69% vaccine activity loss.

### EXAMPLE 15

A powder mixture containing 3.97% EDTA, 5.29% monobasic potassium phosphate, 43.64% dibasic potassium phosphate, 1.85% sodium thiosulfate, 11.37% sodium chloride, 31.74% lactose, 0.60% magnesium stearate, 0.60% silicon dioxide and 0.03% colorant. This mixture is metered at a ratio of 3.78 g per each liter of water to prepare.

A laboratory scale test was conducted in which the product was applied to a water blank solution having pH 6, 100 ppm total hardness and 5 ppm free chlorine, and within five minutes a vial having 1000 doses with a titer of 3.7 log 10 of vaccine against avian infectious bronchitis disease which was used as biological model (sample A) and a duplicate sample with the blank solution without the product (sample B) was also applied.

After 2 hours of mixing the following physiochemical measurements by instrumental analysis and biological measurements by chick embryo titration were obtained:

Sample A: water hardness of 2 ppm total hardness, a pH of 7.0 and a zero free chlorine titration. It was observed that the vaccine titer was kept at a concentration of 97% of the initial applied concentration of the vaccine so that there was only 3% of vaccine activity loss.

Sample B: a hardness of 100 ppm, pH of 6.0 and 5 ppm of free chlorine were obtained. It was observed that the vaccine titer was reduced to 25% of the initial applied concentration of the vaccine so there was a 75% vaccine activity loss.

### EXAMPLE 16

A powder mixture containing 2.21% EDTA, 2.95% monobasic potassium phosphate, 24.34% dibasic potassium phosphate, 1.03% sodium thiosulfate, 6.34% sodium chloride, 17.70% lactose, 0.59% magnesium stearate, 0.59% silicon dioxide and 44.25% colorant. This mixture is metered at a ratio of 6.78 g per each liter of water to prepare.

A laboratory scale test was conducted in which the product was applied to a water blank solution having pH 6, 100 ppm total hardness and 5 ppm free chlorine, and within five minutes a vial having 1000 doses with a titer of 3.7 log 10 of vaccine against avian infectious bronchitis disease which was used as biological model (sample A) and a duplicate sample with the blank solution without the product (sample B) was also applied.

After 2 hours of mixing the following physiochemical measurements by instrumental analysis and biological measurements by chick embryo titration were obtained:

Sample A: water hardness of 35 ppm total hardness, a pH of 6.9 and a zero free chlorine titration. It was observed that the vaccine titer was kept at a concentration of 92% of the initial applied concentration of the vaccine so that there was only 8% of vaccine activity loss.

Sample B: a hardness of 109 ppm, pH of 6.0 and 5 ppm of free chlorine were obtained. It was observed that the vaccine titer was reduced to 33% of the initial applied concentration of the vaccine so there was a 67% vaccine activity loss.

Having described the invention, it is considered as novelty in the field of application and therefore claimed as exclusive property the contents of the following claims:

1. Vaccine stabilizing formula with live antigens for use in mass vaccination systems, characterized in that it comprises a buffer solution and a reducing agent.

2. Vaccine stabilizing formula with live antigens for use in mass vaccination systems according to claim 1, characterized in that the buffer solution is made up of a pH stabilizing agent and a water hardness sequestrant agent.

3. Vaccine stabilizing formula with live antigens for use in mass vaccination systems according to claims 1 and 2, characterized in that the pH stabilizing agent is one of the following compounds: phosphates, succinates, bicarbonates, acetates and lactates, among others; preferably monobasic potassium phosphate and dibasic potassium phosphate.

4. Vaccine stabilizing formula with live antigens for use in mass vaccination systems according to claims 1 and 2, characterized in that the water sequestrant agent is one of the following compounds: ethylene diaminetetraacetic acid (EDTA) monosodium salt, ethylene diaminetetraacetic acid (EDTA) disodium salt, ethylene diaminetetraacetic acid (EDTA) trisodium salt and ethylene diaminetetraacetic acid (EDTA) tetrasodium salt, among others; preferably ethylene diaminetetraacetic acid (EDTA) disodium salt.

5. Vaccine stabilizing formula with live antigens for use in mass vaccination systems according to claim 1, characterized in that the reducing agent is one of the following compounds: sodium thiosulfate, sodium metabisulfite, sodium bisulfite, sodium sulfite, sulphur dioxide, ammonium bisulfite, and ammonium thiosulfate, among others, preferably sodium thiosulfate.

6. Vaccine stabilizing formula with live antigens for use in mass vaccination systems according to claims 1 to 5, characterized in that there can optionally be added one or any combination of the following compounds:
a. a water soluble salt, such as chlorides, iodides, carbonates, bicarbonates, phosphates, iodates, chlorates, bromides, bromates, fluorides, nitrates, nitrites, sulfides, sulfates and sulfites, among others; preferably sodium chloride, in order to provide the necessary osmotic pressure for keeping stable the virus structure and the bacteria outer wall;
b. a carbohydrate, such as glucose, dextrose, lactose, sucrose, mannose and fructose, among others; preferably lactose, for protecting the virus or bacterium structure from the attack of poultry digestive tract adverse conditions;
c. a food grade colorant, preferably brilliant blue color, for providing the user a visual verification medium in order to determine the moment at which the stabilizer was applied and the animals that received the dose;
d. a food grade anti-wetting agent, such as silicon dioxide, calcium stearate, magnesium stereate, tribasic calcium phosphate, tribasic magnesium phosphate, magnesium oxide, calcium silicate, magnesium silicate, sodium silico aluminate, calcium silicate aluminate, among others; preferably silicone dioxide and magnesium stearate, in order to prevent wetting of the mixture as it has hygroscopic properties when contacting the environment.

7. Vaccine stabilizing formula with live antigens for use in mass vaccination systems according to claims 1 to 6, characterized in that said ethylene diaminetetraacetic acid (EDTA) disodium salt is at a range of 0.03% to 34.19%, said monobasic potassium phosphate at a range of 0.03% to 34.48%; said dibasic potassium phosphate at a range of 0.03% to 56.07%; said sodium thiosulfate at a range of 0.03% to 33.73%; said sodium chloride at a range of 0.03% to 35.91%; said lactose at a range of 0.03% to 51.02%; said silicon dioxide at a range of 0.03% to 5%; said magnesium stearate at a range of 0.03% to 5% and said brilliant blue colorant at a range of 0.03% to 44.25%.

8. Vaccine stabilizing formula with live antigens for use in mass vaccination systems according to claim 7, characterized in that the optimal concentration of said ethylene diaminetetraacetic acid (EDTA) disodium salt is of 3.75%, of said monobasic potassium phosphate of 5%, of said dibasic potassium phosphate of 41.25%, of said sodium thiosulfate of 1.75%, of said sodium chloride of 10.75%, of said lactose of 28%, of said silicon dioxide of 1%, of said magnesium stearate of 1% and of said brilliant blue colorant of 7.50%.
